(19) United States
(12) Patent Application Publication
(10) Pub. No.: US 2006/0094002 A1
(43) Pub. Date: May 4, 2006

(54) Eukaryotic biosensor making use of a calcium regulated light emitting enzyme

(76) Inventor: Olga Kozlova-Zwinderman, Edinburgh (GB)

Correspondence Address: FLESHNER & KIM, LLP, P.O. BOX 221200, CHANTILLY, VA, 20153, US

(21) Appl. No.: 10/537,435

(22) PCT Filed: Dec. 2, 2003
(86) PCT No.: PCT/GB03/05272

§ 371 Date: Nov. 23, 2005

(30) Foreign Application Priority Data
Dec. 3, 2002 (GB) 0228139.2

(51) Int. Cl. C12Q 1/00; C12N 1/16

(52) U.S. Cl. 435/4; 435/254.3

(57) ABSTRACT

The present invention provides a method of using eukaryotic cells being transformed with a light emitting Ca2+ regulated photoprotein gene for determining the presence or absence of at least one toxic substance in a sample and for assisting in the identification of the toxicant(s). More specifically there is provided a toxicity assay for various uses including determining the presence of toxins and in particular heavy metals and organophenols, general cytoxicity testing of pure chemicals and chemical mixtures in particular for drug development testing, testing of food and drink products, cosmetics testing and identification of organisms in particular of fungal strains.

The present invention provides a method of using transformed eukaryotic cells or organisms for determining the presence or absence of at least one toxic substance in a sample and for assisting in the identification of the toxicant(s). More specifically there is provided a toxicity assay for various uses including determining the presence of toxins, general cytotoxicity testing of pure chemicals and chemical mixtures in particular for drug development testing, testing of food and drink products, cosmetics testing and identification of organisms in particular of fungal strains

The release of contaminating substances into an environment such as a waterway or an area of agricultural land can have serious effects on the ecosystems found in that environment. It is important to be able to analyse these effects both prior to the release of such contaminants so as to manage their treatment or release, and after release so as to determine and counteract their effects.

Current methods used to monitor water quality and screen effluent generally involve chemical toxicity tests. However, these tests require a general idea of the type of contaminant being tested for and can be very expensive.

Similarly the presence of contaminating substances or toxins can be problematic in other areas such as food and drink manufacture and cosmetics manufacture. There are also instances, such as in drug development and cosmetic industry, where the substance of interest i.e. the potential new drug may itself be a contaminating substance or toxin and this needs to be checked.

Biosensors are used for toxicity testing and are well known in the field. Toxicity depends on a variety of factors including pH, temperature, salinity and contaminant concentration, but depends especially on the test organism used in the sensor.

One of the most commonly used organisms is the bioluminescent bacterium, Vibrio fischeri. The bioluminescence involved is mediated by the luciferin-luciferase enzyme system wherein light emission is dependent on the electron transfer chain. Any disruption to the electron transfer chain, for example on exposure to a toxicant, affects light emission. Light emission at the time a substance is added is therefore indicative of the presence of a toxic substance.

This system, however, only provides a simple indication of whether a contaminant is toxic or not. No detailed information is obtained on how toxic the contaminant is, nor is the contaminant identified.

The terms toxicant and toxin as herein described relate to compounds, chemicals and mixtures of chemicals which have an effect on eukaryotic cells or organisms and in particular which are toxic to eukaryotic organisms such as fungus or which have anti-fungal activity.

The term eukaryote as herein described relates to eukaryotic cells or organisms.

According to a first aspect of the present invention there is provided a method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
determining whether the amount of light is above or below a defined threshold at the time of exposure.

Optionally the eukaryote is a fungi. (throughout this document fungi should be considered under its typical classification as covering both multicellular organisms and unicellular organisms such as the yeast Saccharomyces cerviseae Preferably the fungi is a filamentous fungi.

More preferably the fungi is of the Aspergillus species.

Alternatively the eukaryote is a mammalian cell.

A further alternative is that the eukaryote is a plant cell.

Preferably the test sample comprises a toxicant.

Preferably the light emitting Ca²⁺ regulated photoprotein gene is a recombinant gene.

Preferably the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

Optionally, the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

Most preferably the light emitting Ca²⁺ regulated photoprotein gene is an aequorin gene.

More preferably the light emitting Ca²⁺ regulated photoprotein gene is a recombinant aequorin gene.

Preferably the light that is measured is in the form of luminescence.

Optionally the test sample is added in advance of the application of a stimulus to the test sample.

Preferably the stimulus is at least one or more from the group comprising; mechanical perturbation, hypo-osmotic shock, and change in external calcium chloride concentration, temperature shock, pH shock.

Preferably the test sample is added 1 minute to 1 hour prior to the application of the stimulus.

More preferably the test sample is added 5 minutes prior to the application of the stimulus.

More preferably the test sample is added 30 minutes prior to the application of the stimulus.

According to a second aspect of the present invention there is provided a method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
determining whether the amount of light is above a defined threshold at a specified time after the time of exposure.

Optionally the method comprises the step of determining whether the amount of light is below a defined threshold.

Optionally the specified time after the time of exposure is 11 minutes.

Optionally the eukaryote is a fungi.

Preferably the fungi is a filamentous fungi.

More preferably the fungi is of the Aspergillus species.

Alternatively the eukaryote is a mammalian cell.

A further alternative is that the eukaryote is a plant cell.

Preferably the test sample comprises a toxicant.

Preferably the light emitting Ca²⁺ regulated photoprotein gene is a recombinant gene.

Preferably the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

Optionally, the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

Most preferably the light emitting Ca²⁺ regulated photoprotein gene is an aequorin gene.

More preferably the light emitting Ca²⁺ regulated photoprotein gene is a recombinant aequorin gene.

Preferably the light that is measured is in the form of luminescence.

Optionally the test sample is added in advance of the application of a stimulus to the test sample.

Preferably the stimulus is at least one or more from the group comprising; mechanical perturbation, hypo-osmotic shock, change in external calcium chloride concentration, temperature shock, pH shock.

Preferably the test sample is added 1 minute to 1 hour prior to the application of the stimulus.

More preferably the test sample is added 5 minutes prior to the application of the stimulus.

More preferably the test sample is added 30 minutes prior to the application of the stimulus.

According to a third aspect of the present invention there is provided a method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
and comparing at least one parameter of the light measurement data with a bank of known toxicity reference data.

Optionally the method comprises the step of determining whether the amount of light is below a defined threshold.

Optionally the specified time after the time of exposure is 11 minutes.

Optionally the eukaryote is a fungi.

Preferably the fungi is a filamentous fungi.

More preferably the fungi is of the Aspergillus species.

Do we need next two sentences

Most preferably the fungi is Aspergillus awamori.

Most preferably the strain of Aspergillus awamori is strain 66A.

Alternatively the eukaryote is a mammalian cell.

A further alternative is that the eukaryote is a plant cell.

Preferably the test sample comprises a toxicant.

Preferably the light emitting Ca²⁺ regulated photoprotein gene is a recombinant gene.

Preferably the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

Optionally, the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

Most preferably the light emitting Ca²⁺ regulated photoprotein gene is an aequorin gene.

More preferably the light emitting Ca²⁺ regulated photoprotein gene is a recombinant aequorin gene.

Preferably the light that is measured is in the form of luminescence.

Optionally the test sample is added in advance of the application of a stimulus to the test sample.

Preferably the stimulus is at least one or more from the group comprising; mechanical perturbation, hypo-osmotic shock, change in external calcium chloride concentration, temperture shock, pH shock.

Preferably the test sample is added 1 minute to 1 hour prior to the application of the stimulus.

More preferably the test sample is added 5 minutes prior to the application of the stimulus.

More preferably the test sample is added 30 minutes prior to the application of the stimulus.

Preferably, the method is used to determine the amount of toxicant in the sample.

Optionally, the method is used to identify the toxicant in the sample.

According to a fourth aspect of the present invention there is provided a method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
converting the light data into a cytosolic free calcium ion concentration trace,
and comparing at least one parameter of the cytosolic free calcium ion concentration trace with a bank of known toxicity reference data.

Optionally the method comprises the step of determining whether the amount of light is below a defined threshold.

Optionally the specified time after the time of exposure is 11 minutes.

Optionally the eukaryote is a fungi.

Preferably the fungi is a filamentous fungi.

More preferably the fungi is of the Aspergillus species.

Alternatively the eukaryote is a mammalian cell.

A further alternative is that the eukaryote is a plant cell.

Preferably the test sample comprises a toxicant.

Preferably the light emitting Ca²⁺ regulated photoprotein gene is a recombinant gene.

Preferably the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

Optionally, the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

Most preferably the light emitting Ca²⁺ regulated photoprotein gene is an aequorin gene.

More preferably the light emitting Ca²⁺ regulated photoprotein gene is a recombinant aequorin gene.

Preferably the light that is measured is in the form of luminescence.

Optionally the test sample is added in advance of the application of a stimulus to the test sample.

Preferably the stimulus is at least one or more from the group comprising; mechanical perturbation, hypo-osmotic shock, change in external calcium chloride concentration, temperature shock, pH shock.

Preferably the test sample is added 1 minute to 1 hour prior to the application of the stimulus.

More preferably the test sample is added 5 minutes prior to the application of the stimulus.

More preferably the test sample is added 30 minutes prior to the application of the stimulus. Preferably light is measured for between 1 minute and 5 hours following the application of the stimulus.

More preferably light is measured for 5 minutes following the application of the stimulus.

Preferably, the cytosolic free calcium ion trace is a plot of the cytosolic free calcium ion concentration against time.

Preferably the parameter is at least one or more selected from the group comprising;
lag time
rise time
absolute amplitude
relative amplitude
Length of transient
number of cytosolic free calcium ion concentration increases
percentage increase in final cytosolic free calcium ion concentration resting level
percentage increase in recovery time
percentage increase in pre-stimulating cytosolic free calcium ion concentration resting level
Total concentration of Ca²⁺ released.

Preferably, the method is used to determine the amount of toxicant in the sample.

Optionally, the method is used to identify the toxicant in the sample.

According to a fifth aspect of the present invention there is provided an assay for use in determining the presence of a known toxicant in a test sample, the assay comprising the steps of;
exposing a fungi transformed with a recombinant aequorin gene to a test sample of a substance,
measuring the luminescence produced by the fungi,
converting the luminescence data into a cytosolic free calcium ion concentration trace,
and comparing at least one parameter of the cytosolic free calcium ion concentration trace with a bank of known toxicity reference data.

Preferably the cytosolic free calcium ion trace is a plot of the cytosolic free calcium ion concentration against time.

Preferably the fungi transformed with a recombinant aequorin gene is a filamentous fungi.

More preferably the fungi is of the Aspergillus species.

Preferably the substance is a contaminant.

Preferably the substance is a contaminated sample.

Preferably the parameter is at least one or more selected from the group comprising; lag time, rise time, absolute amplitude, relative amplitude, length of transient at 20%, 50% and 80% of maximum amplitude, number of cytosolic free calcium ion concentration increases, percentage increase in final cytosolic free calcium ion concentration resting level, percentage increase in recovery time and percentage increase in the total amount of Ca²⁺ released.

Optionally, the test sample is added in advance of the application of a stimulus to the test sample.

Preferably the stimulus is at least one or more from the group comprising; mechanical perturbation, hypo-osmotic shock, change in external calcium chloride concentration, temperature shock and pH shock.

Preferably the test sample is added 1 minute to 1 hour prior to the application of the stimulus.

More preferably the test sample is added 5 minutes prior to the application of the stimulus.

More preferably the test sample is added 30 minutes prior to the application of the stimulus.

In such instances, the parameters may include at least one or more selected from the group comprising; lag time, rise time, absolute amplitude, relative amplitude Length of transient at 20%, 50% and 80% of maximum amplitude, number of cytosolic free calcium ion concentration increases, percentage increase in final cytosolic free calcium ion concentration resting level, percentage increase in recovery time, percentage increase in pre-stimulating cytosolic free calcium ion concentration resting level and percentage increase in the total amount of Ca²⁺ released.

Preferably luminescence is measured for between 1 minute and 5 hours following the application of the stimulus.

More preferably luminescence is measured for 5 minutes following the application of the stimulus.

Preferably, the method is used to determine the amount of toxicant in the sample.

Optionally, the method is used to identify the toxicant in the sample.

In order to further explain the present invention details of a number of experiments are provided.

A first experiment comprises testing the effect of pre-incubation of Aspergillus awamori with toxicants on cytosolic free calcium ion concentration response to an increase in external calcium chloride.

A further set of experiments described herein shows attempts to obtain characteristic data for a range of different toxicants at a number of different concentrations. The results demonstrate that each toxicant at each concentration produces a distinctive cytosolic free calcium ion concentration trace whose traits could be used to identify and characterise a toxicant present in a test sample.

A final experiment attempts to determine whether it is possible to identify and characterise individual toxicants from testing samples of mixtures of toxicants in different proportions. The traces produced are distinct for each mixture.

These results show that it is possible to characterise and identify a specific toxicant from a test sample by using the characteristic data obtained from a cytosolic free calcium ion concentration trace.

It is also possible to characterise and identify a specific toxicant from a test sample by using the characteristic data obtained from light readings. The main difference between doing light emission and cytosolic free calcium ion concentrations is the removing the step of converting the luminescence data into a cytosolic free calcium ion concentration trace”.

So the Method is:

An assay for use in determining the presence of a known toxicant in a test sample, the assay comprising the steps of;
exposing a fungi transformed with a recombinant aequorin gene to a test sample of a substance,
measuring the luminescence produced by the fungi in relative light units (RLU),
and calculating the following parameters: lag time, rise time, length of transient (LT₂₀, LT₅₀, LT₈₀), absolute amplitude, relative amplitude, recover time, final level of luminescence, initial level of luminescence, total luminescence.

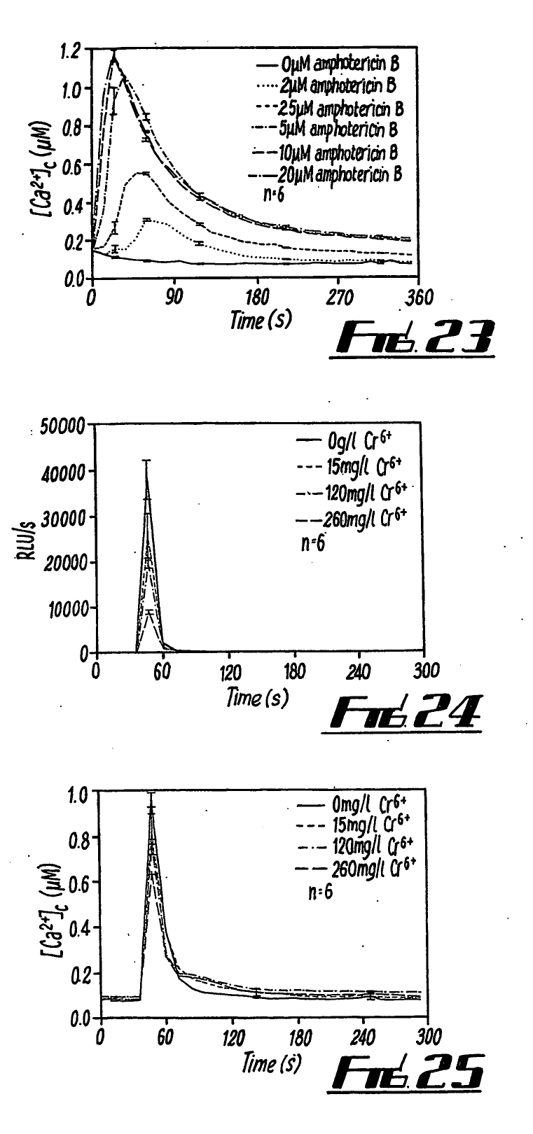

Since RLU are not normalised with regard to the biomass, the parameters measured in relative light units (RLU) are different from the cytosolic free calcium ion concentration [Ca²⁺]. FIGS. 24 and 25 show that the decrease in amplitude caused by 260 mg/l Cr⁶⁺ is 75% in RLU, and only 65% in Ca²⁺ concentration. Other parameters would differ in a similar way.

Most of the toxicity testing for environmental pollutants is usually carried out using RLU and therefore the light-emitting essay would be particularly helpful if used alongside other existing biosensors.

The parameters referred to herein relate to the following;

Lag Time, the time from addition of the test sample to the time when the cytosolic free calcium ion concentration, [Ca²⁺]_{c}, began to rise;

Rise Time, the time from addition of the test sample to the time at which maximum [Ca²⁺]_{c} was reached;

Number of [Ca²⁺]_{c} Rises, the number of peaks in [Ca²⁺]_{c};

Percentage Increase in Final [Ca²⁺ c Resting Level, the percentage increase in resting [Ca²⁺]_{c} at the end of the experiment, where the control value is taken to be 100%;

Percentage Increase in Recovery Time, percentage increase in recovery time where recovery time represents the total amount of [Ca²⁺]_{c} released during the period of time from the point when the maximum amplitude following calcium chloride treatment was achieved to the point when the [Ca²⁺]_{c} reached its final resting level. Recovery time was initially calculated for control cultures. In the control this period of time was calculated as 250 seconds. For the cultures subjected to the treatment with toxicant(s) the total amount of [Ca²⁺]_{c} was calculated for the same period of 250 seconds starting from the maximum amplitude. The recovery time of the control cultures was therefore:

$$\frac{\begin{matrix}total amount {of [ {Ca}^{2 +} ]}_{c} ( μM ) for the toxicant - \\ treated samples over 250 seconds \times 100\end{matrix}}{\begin{matrix}total amount {of [ {Ca}^{2 +} ]}_{c} ( μM ) for the control sample \\ over 250 seconds\end{matrix}}$$

Percentage Increase in pre-Stimulating [Ca²⁺]_{c} Resting Level, the percentage increase in [Ca²⁺]_{c} prior to the stimulus, where the control value is taken to be 100%.

Percentage change in total amount of calcium released during the transient at stage 1—calculated by integration of the all luminescence obtained after addition of the compounds of interest before subsequent stimulation with physico-chemical stimuli.

Percentage change in total amount of calcium released during the transient at stage 2—calculated by integration of the all luminescence obtained after the fungus is stimulated with one of the physico-chemical stimuli.

Percentage change in total amount of calcium released during the whole transient—calculated by integration of the all luminescence obtained during the period of experiment.

Length of transient (LT)—this parameter describes the length of the transient when the amplitude of the response is equal a certain percentage from the maximum amplitude.

LT₂₀ (Length of transient at Amplitude=20% of maximum Amplitude)

LT₅₀ (Length of transient at Amplitude=50% of maximum Amplitude)

LT₈₀ (Length of transient at Amplitude=80% of maximum Amplitude)

All secondary increases have to be analysed by the same parameters as primary increases during stages 1 and 2.

E.g. Amplitude, length, rise time, lag time,

Percentage change in amplitude should be assessed as the absolute value from point 0 (Aₐ) and as the relative value from the initial resting level (Aᵣ). The relative changes assess the ability of of the eukaryote to respond to the physiological stimuli. This parameter is important to assess the physiological state of the eukaryote.

There is also the possibility of combining one or more of these parameters to obtain further values which can be used for identification of the toxicants in the mixture. For example, the summation of amplitude and recovery time will give the value of total cytosolic free calcium ions emitted from the time when [Ca²⁺]_{c} reaches its peak. Also summation of lag time and rise time will give the total time required for [Ca²⁺]_{c} to reach its peak. The division of final [Ca²⁺]_{c} resting level onto the pre-stimulation [Ca²⁺]_{c} resting level will show how many times the [Ca²⁺]_{c} resting level has changed after stimulation. Similarly, a division of the final [Ca²⁺]_{c} resting level onto the initial [Ca²⁺]_{c} resting level prior to the addition of toxicant(s) gives further identifying data. Additionally, the summation of all the data points of the trace gives the total amount of cytosolic free calcium ions released during the monitoring period.

As mammalian cells are more complex than other eukaryotes such as fungi or plants typically more parameters will be considered.

The present invention will now be described with reference to the following non-limiting examples and with reference to the figures, wherein:

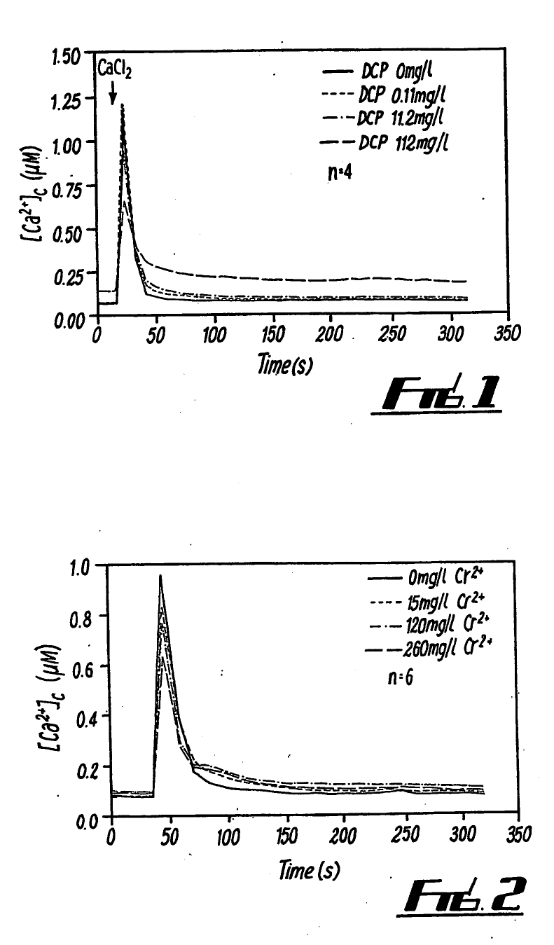

FIG. 1 shows the characteristic [Ca²⁺]_{c} trace produced on addition of 5 mM external CaCl₂, following a 5 minute pre-incubation with different concentrations of 3,5-DCP.

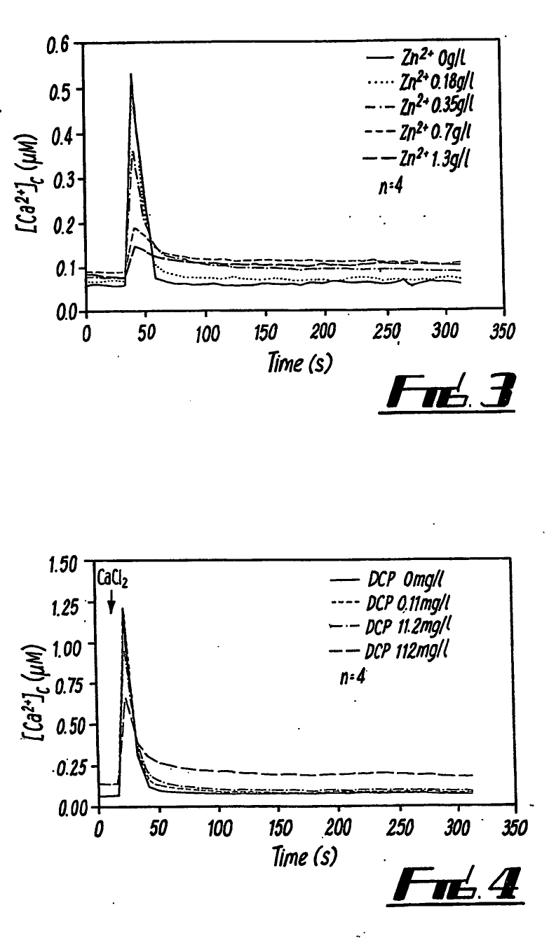

FIG. 2 shows the characteristic [Ca²⁺]_{c} trace produced on addition of 5 mM external CaCl₂, following a 5 minute pre-incubation with different concentrations of Cr⁶⁺.

FIG. 3 shows the characteristic [Ca²⁺]_{c} trace produced on addition of 5 mM external CaCl₂, following a 5 minute pre-incubation with different concentrations of Zn²⁺.

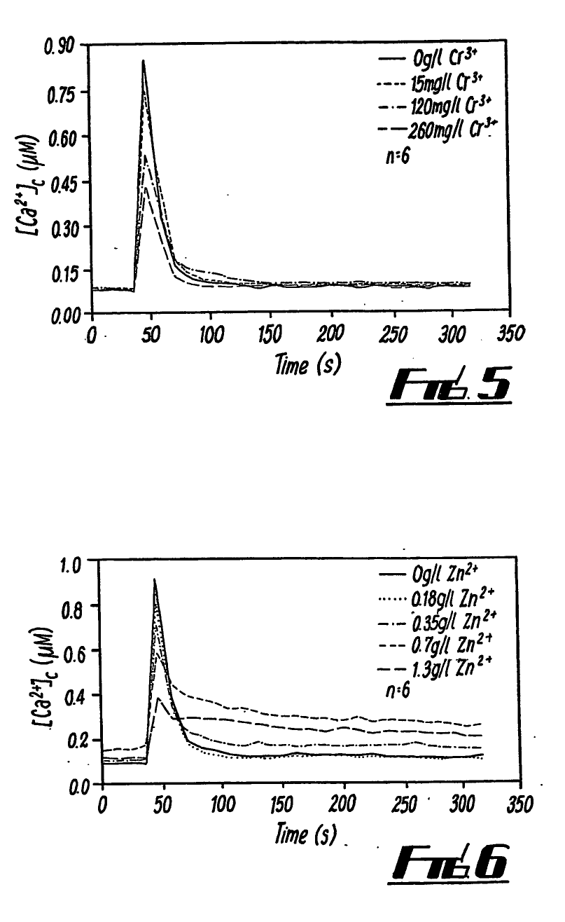

FIG. 4 shows the characteristic [Ca²⁺]_{c} trace produced on addition of 5 mM external CaCl₂, following a 30 minute pre-incubation with different concentrations of 3,5-DCP.

FIG. 5 shows the characteristic [Ca²⁺]_{c} trace produced on addition of 5 mM external CaCl₂, following a 30 minute pre-incubation with different concentrations of Cr⁶⁺.

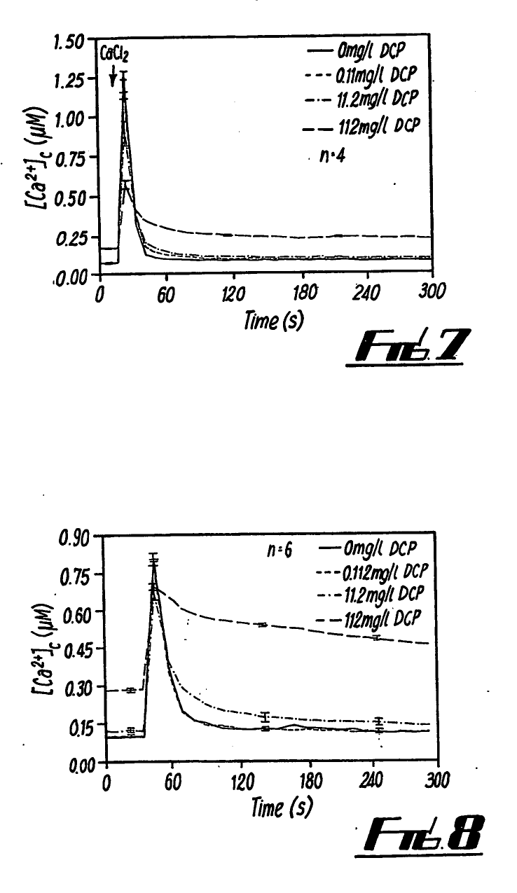

FIG. 6 shows the characteristic [Ca²⁺]_{c} trace produced on addition of 5 mM external CaCl₂, following a 30 minute pre-incubation with different concentrations of Zn²⁺.

FIG. 7 shows the characteristic cytosolic free calcium ion concentration, [Ca²⁺]_{c}, trace produced on addition of 5 mM CaCl₂ following a 5 minute pre-incubation with different concentrations of 3,5-dichlorophenol, 3,5-DCP.

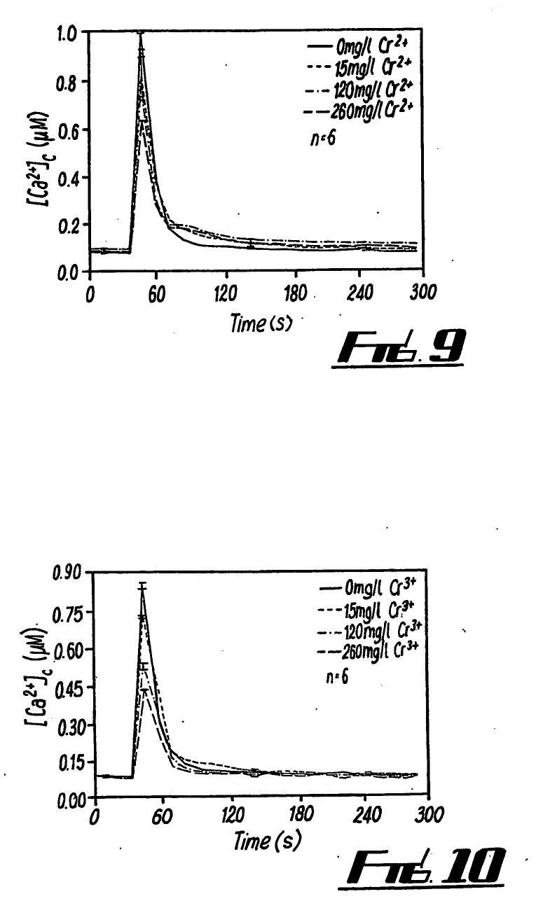

FIG. 8 shows the characteristic [Ca²⁺]_{c} trace produced on addition of 5 mM CaCl₂, following a 30 minute pre-incubation with different concentrations of 3,5-DCP.

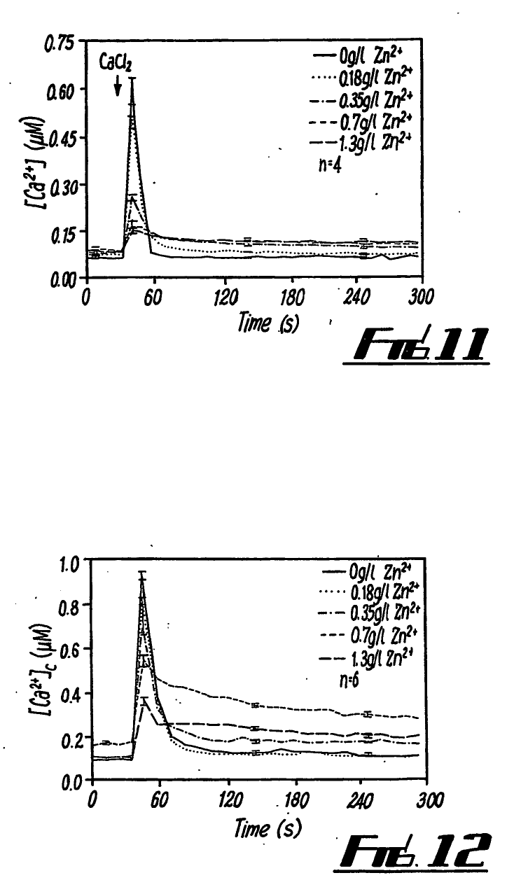

FIG. 9 shows the characteristic [Ca²⁺]_{c} trace produced on addition of 5 mM CaCl₂, following a 5 minute pre-incubation with different concentrations of chromium ions, Cr⁶⁺.

FIG. 10 shows the characteristic [Ca²⁺]_{c} trace produced on addition of 5 mM CaCl₂, following a 30 minute pre-incubation with different concentrations of chromium ions, Cr⁶⁺.

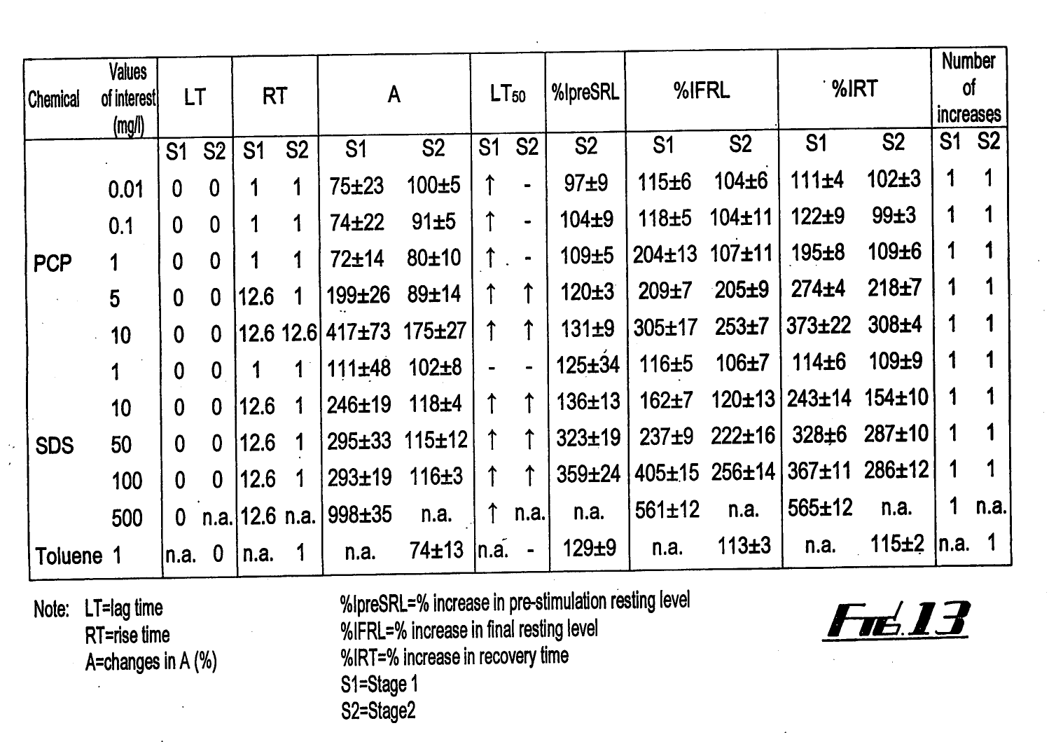

FIG. 11 shows the characteristic [Ca²⁺]_{c} trace produced on addition of 5 mM CaCl₂, following a 5 minute pre-incubation with different concentrations of zinc ions, Zn²⁺.

FIG. 12 shows the characteristic [Ca²⁺]_{c} trace produced on addition of 5 mM CaCl₂, following a 30 minute pre-incubation with different concentrations of zinc ions, Zn²⁺.

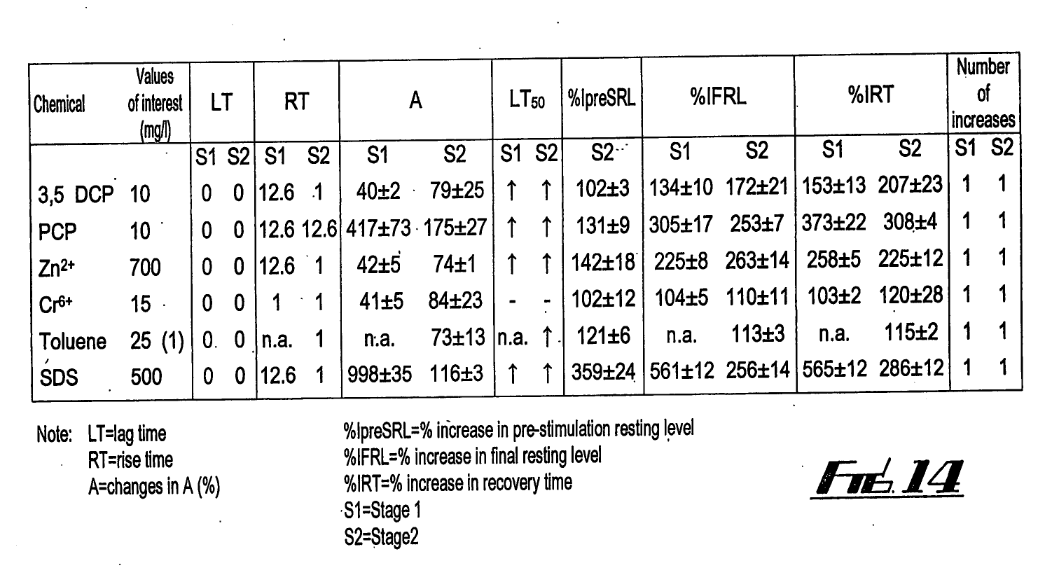

FIG. 13 shows the values of [Ca²⁺]_{c} trace parameters characteristic for different concentrations of pentochlorophenol, PCP; sodium dodecyl sulphate, SDS; and Toluene. Parameters assessed are Lag Time, LT; Rise Time, RT; Amplitude, A; Length of transient, LT50; Percentage Increase in pre-Stimulating [Ca²⁺]_{c} Resting Level, % IpreSRL; Percentage Increase in Final [Ca²⁺]_{c} Resting Level, % IFRL; Percentage Increase in Recovery Time, % IRT; and Number of [Ca²⁺]_{c} Increases.

FIG. 14 shows the values of [Ca²⁺]_{c} trace parameters characteristic for 3,5-DCP, PCP, Zn²⁺, Cr⁶⁺, Toluene, and SDS. Parameters assessed are Lag Time, LT; Rise Time, RT; Amplitude, A; Length of transient, LT50; Percentage Increase in pre-Stimulating [Ca²⁺]_{c} Resting Level, % IpreSRL; Percentage Increase in Final [Ca²⁺]_{c} Resting Level, % IFRL; Percentage Increase in Recovery Time, % IRT; and Number of [Ca^{2+]}_{c} Increases.

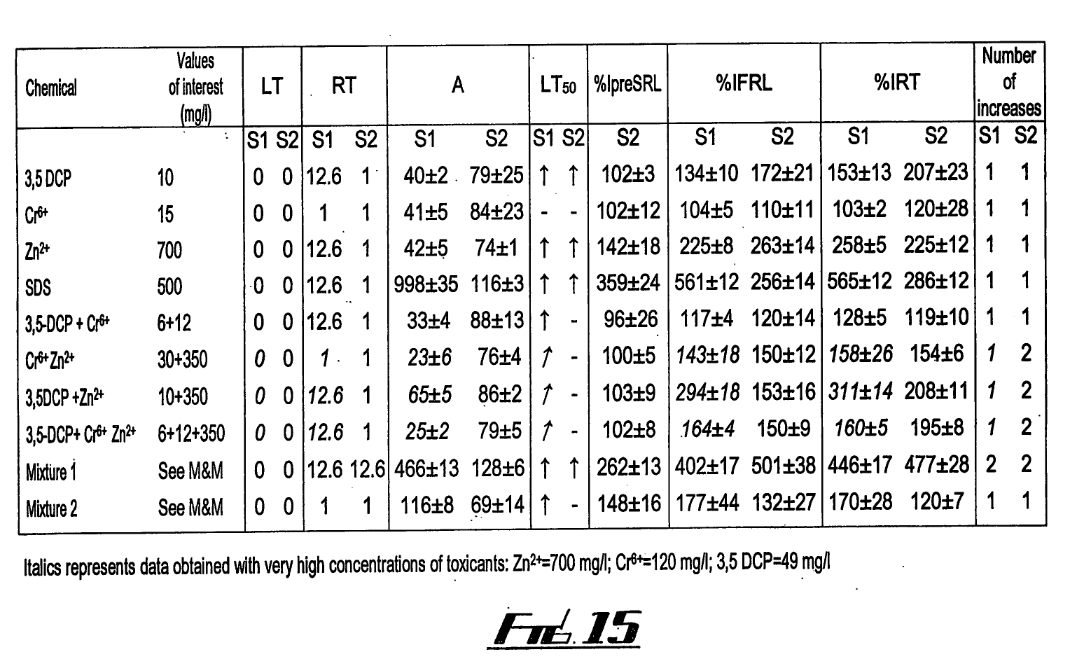

FIG. 15 shows the values of [Ca²⁺]_{c} trace parameters characteristic for different mixtures of toxicants. Parameters assessed are Lag Time, LT; Rise Time, RT; Amplitude, A; Length of transient, LT50; Percentage Increase in pre-Stimulating [Ca²⁺]_{c} Resting Level, % IpreSRL; Percentage Increase in Final [Ca²⁺]_{c} Resting Level, % IFRL; Percentage Increase in Recovery Time, % IRT; and Number of [Ca²⁺]_{c} Increases.

Effect Of Pre-Incubation of Aspergillus Awamori with Toxicants on [Ca²⁺]_{c} Response to External Calcium Chloride

12 ml of sterile VS medium was inoculated with 1×10⁵ spores per ml A. awamori strain 66A. 100 μl of the inoculated medium was added to each well of a 96-well plate and cultured in a humidity chamber in the presence of free water at 30° C. for 24 hours.

The following toxicants were tested: 3,5-dichlorophenol, zinc sulphate, and potassium dichromate. Each toxicant was added in a total volume of 25 μl VS medium or water 5 or 30 minutes before addition of 5 mM calcium chloride.

Luminescence was monitored for 5 minutes following addition of CaCl₂. Aequorin was completely discharged by adding 3M calcium chloride in 20% ethanol. The total concentration is thus 1.5 M calcium chloride in 10% ethanol.

Luminometry was performed using an EG & G Berthold (Bad Wildbad, Germany) LB96P Microlumat luminometer. Luminescence data was converted from real light units to [Ca²⁺]_{c} values using the following equation:
PCa=0.332588 (−log k)+5.5593,
where k=luminescence counts per second/total luminescence counts. Total luminescence is measured as an integral of all luminescence up to complete aequorin discharge.

The Equation is first described in Fricker, M. D., Plieth, C., Knight, H., Blancaflor, E., Knight, M. R., White, N. S., and Gilroy, S. 1999. Fluorescence and Luminescence Techniques to Probe Ion Activities in Living Plant Cells. In Mason, W. T., editor, Fluorescent and Luminescent Probes. Academic Press. London. pp. 569-596.

The following parameters were assessed:

Rise Time, Amplitude, Length of transient, LT50 and Final [Ca²⁺]_{c} Resting Level.

Effects of Different Concentrations of Toxicants on [Ca²⁺]_{c} Traces

Aspergillus awamori were transformed with an expression vector (pAEQ1-15) comprising a gene for synthetic apoaequorin (aeqS) under the control of the constitutive glucose-6-phosphate dehydrogenase promoter (gpdA).

These transformants were cultured in 100 μl of Vogel's medium with 1% sucrose (VS medium) in microwell plates for 24 hours before addition of a toxicant or a control of distilled water. Toxicants were dissolved in water to give the concentrations shown below. 25 μl of the each of the following concentrations were added to each culture:

| TOXICANT | CONCENTRATIONS (mg/l) |
|---|---|
| 3,5-dichlorophenol (3,5-DCP) | 0.112, 11.2, 112 |
| Chromium ions (Cr⁶⁺) | 15, 120, 260 |
| Zinc ions (Zn²⁺) | 180, 350, 700, 1300 |

The cultures were incubated for 5 or 30 minutes before addition of 100 μl 5 mM CaCl₂. Luminescence was measured for 5 minutes using a plate luminometer. Luminescence data was manually converted from relative light units to cytosolic free calcium ion concentration, [Ca²⁺]_{c}. This was then plotted against time and parameters of this trace were analysed. Parameters assessed were as follows:

Rise Time, the time from addition of CaCl₂ to the moment when maximum [Ca²⁺]_{c} was achieved;

Amplitude, the maximum [Ca²⁺]_{c} reached during the experiment;

Length of transient, at 50% of maximum amplitude the width of the transient at the point where the amplitude equals half of the maximum amplitude of the transient;

and Final Resting [Ca²⁺]_{c} Level, the resting [Ca²⁺]_{c} at the end of the experiment.

Effects of Further Toxicants on [Ca²⁺]_{c} Traces

Cultures of Aspergillus awamori as described above were used to test the effects of further toxicants. The concentrations of toxicants tested were made up as follows in water, where the concentrations tested were based on Dutch target and intervention values for toxicants and Kelly Guidelines for the classification of contaminated soils:

| TOXICANT | CONCENTRATION (mg/l) |
|---|---|
| Pentochlorophenol, PCP | 0.01, 0.1, 1, 5, 10 |
| Sodium dodecyl sulphate, SDS | 1, 10, 50, 100, 500 |
| Toluene | 1, 25 |
| 3,5-DCP | 10 |
| Zn²⁺ | 700 |
| Cr⁶⁺ | 15 |

In the first set-up (S1), 100 μl of each toxicant concentration or of the control (VS medium) were added to the cultures through built-in injectors and luminescence monitored for 5 minutes. In a second set of experiments (S2), cultures were pre-incubated with the toxicant or control for 5 minutes before addition of 5 mM CaCl₂ in a total volume of 25 μl distilled water (pre-incubation can be anywhere between 1 minute and 96 hours). Luminescence was monitored for 5 minutes following addition of CaCl₂. (monitoring can be anywhere between 1 minute and 96 hours). Luminescence data was converted from relative light units to [Ca²⁺]_{c} values as described above. The following parameters were assessed in S1:

Lag Time, the time from addition of CaCl₂ to the time when [Ca²⁺]_{c} began to rise;

Rise Time;

Absolute amplitude;

Relative amplitude

Length of transient (LT20, LT50, LT80);

Percentage Increase in Final [Ca²⁺]_{c} Resting Level, where the control value was taken to be 100%;

Percentage Increase in Recovery Time, where the control value was taken to be 100%; and Number of [Ca²⁺]_{c} Increases, the number of [Ca²⁺]_{c} transients.

Total Ca²⁺ concentration

In S2, the Percentage Increase in pre-Stimulating [Ca²⁺]_{c} Resting Level, where the control value was taken to be 100%, was assessed in addition to all of the parameters tested in S1.

### Effects of Mixtures Containing Different Propotion of Toxicants on [Ca²⁺]_{c} Traces

The experiments described when examining the effects of further toxicants were repeated for different mixtures of toxicants. The following mixtures were made up in water for testing:

6 mg/l 3,5-DCP+12 mg/l Cr⁶⁺

30 mg/l Cr⁶⁺+350 mg/l Zn²⁺

10 mg/l 3,5-DCP+350 mg/l Zn²⁺

6 mg/l 3,5-DCP+12 mg/l Cr⁶⁺+350 mg/l Zn²⁺

Mixture 1: 20 mg/l Cadmium
100 mg/l Copper
50 mg/l Chromium
250 mg/l Zinc
500 mg/l SDS

Mixture 2: 20 mg/l Cadmium
100 mg/l Copper
50 mg/l Chromium
250 mg/l Zinc

These experiments demonstrate a novel finding that each toxicant results in a different and characteristic [Ca²⁺]_{c} transient. Additionally each concentration of toxicant produces a unique [Ca²⁺]_{c} transient. From these characteristic fingerprint responses a profile of data can be built up and used to create a bank of data for each toxicant. Results from testing samples can be compared with this data bank and the presence of a particular toxicant can thus be determined. Furthermore, details such as the mode of action of the toxicant, and the amount of toxicant present can be deduced from a comparison with the bank of pre-gathered data.

Examples of Types of Testing that can be Carried out According to the Present Invention

Specific examples of types of test that can be carried out according to the present invention are given below. Although the tests below describe the use of aequorin expressed fungi according to the present invention, it can be seen that any appropriate eukaryotic cell or organism could be used (i.e. mammalian cells in place of the fungi) which has been transformed with any appropriate gene according to the present invention (i.e. halistaurin in place of aequorin)

The examples refer to the following figures in which;

FIG. 16 shows a graph indicating the effect of 6 environmental samples on [Ca²⁺]_{c};

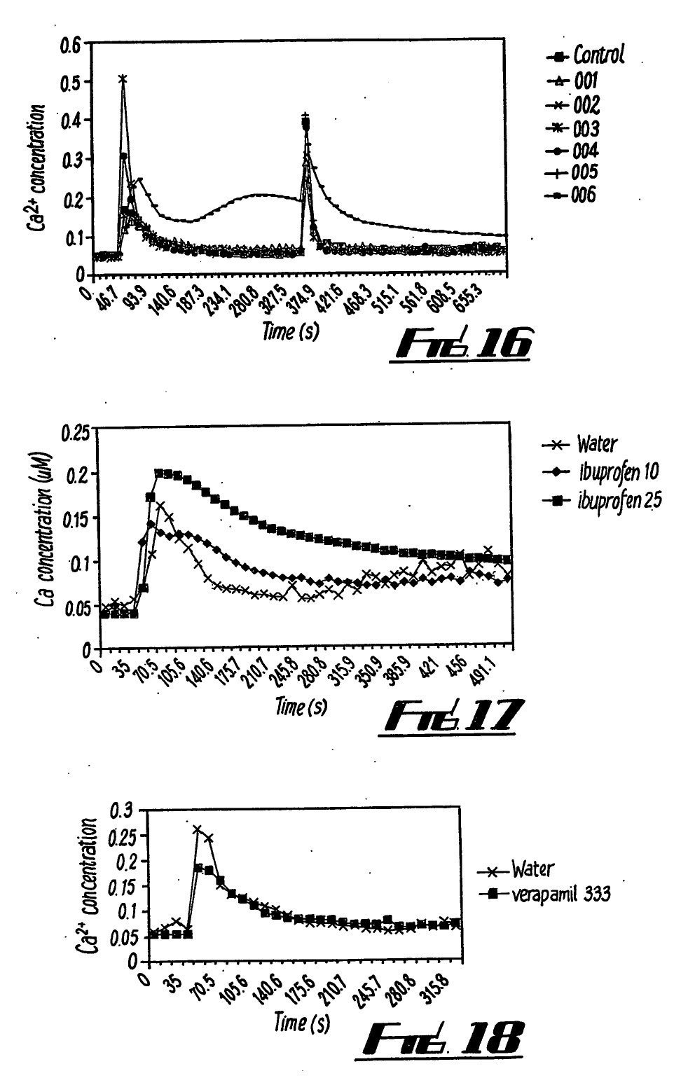

FIG. 17 shows a graph indicating the effect of ibuprofen analogue on [Ca²⁺]_{c};

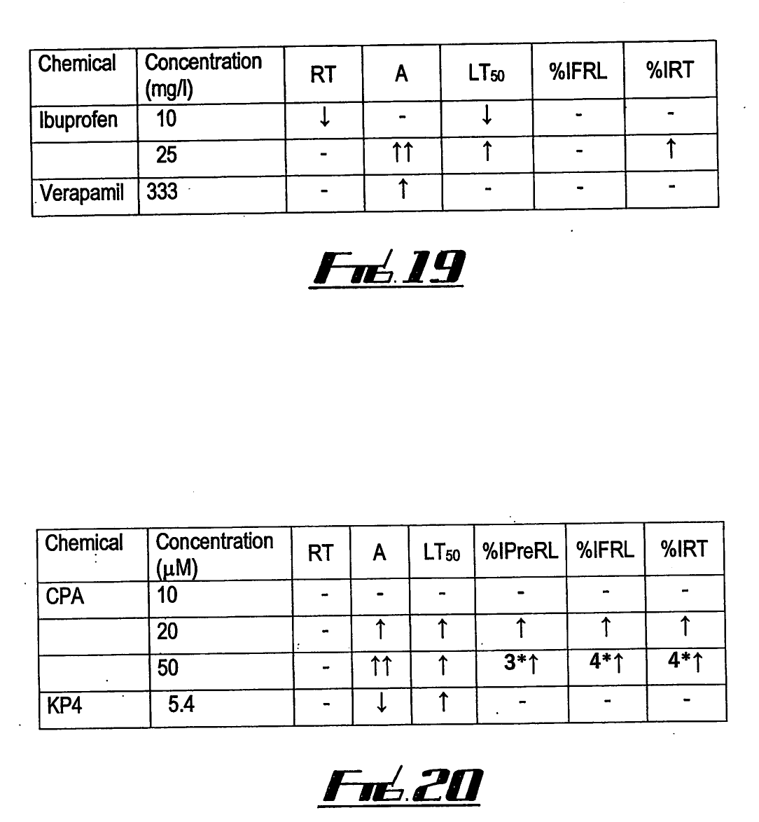

FIG. 18 shows a graph indicating the effect of verpamil on [Ca²⁺]_{c};

FIG. 19 is a table summarising the profiles of the ibuprofen™ ((S)-(−)-o-Acetulmandelic acid) and verapamil™ (Verapamil hydrochloride) analogues;

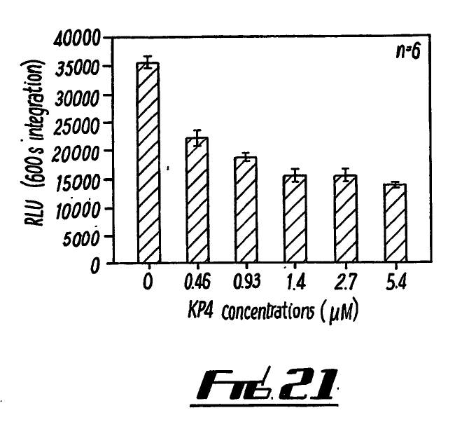

FIG. 20 is a table summarising profiles of cyclopiazonic acid (CPA) and KP4 (mycotoxin produced by Ustilago spp);

FIG. 21 is a graph showing the dose-dependent effect of KP4 on the [Ca²⁺]_{c} response to 5 mM external CaCl₂ (results represent mean±SE);

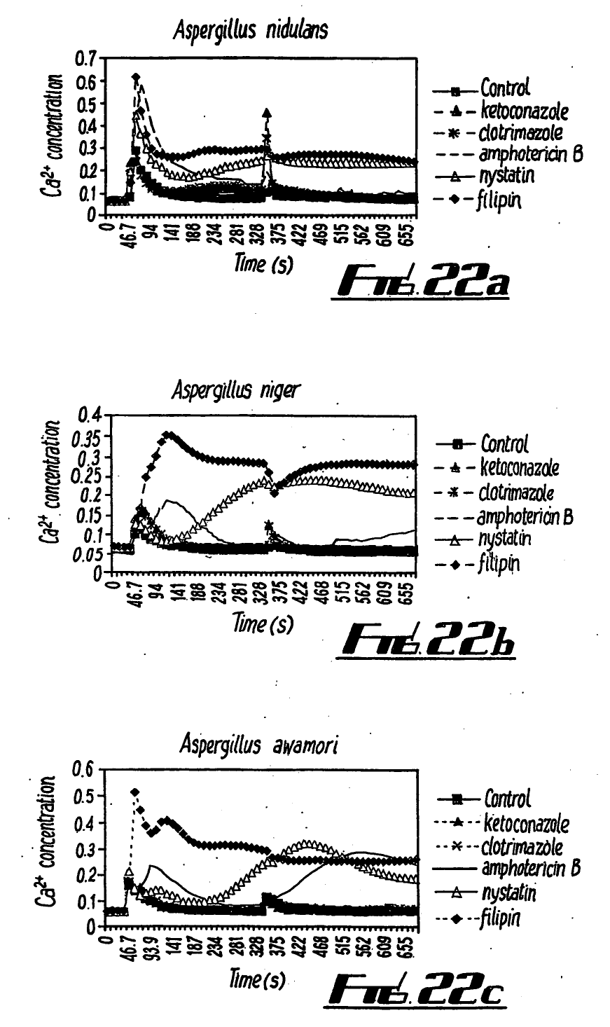

FIG. 22a is a graph showing the effect of known antifungal drugs on [Ca²⁺]_{c} in Aspergillus nidulans;

FIG. 22b is a graph showing the effect of known antifungal drugs on [Ca²⁺]_{c} in Aspergillus niger;

FIG. 22c is a graph showing the effect of known antifungal drugs on [Ca²⁺]_{c} in Aspergillus awamori; and

FIG. 23 is a graph showing the effect of amphotericin B on [Ca²⁺]_{c} (results represent mean±SE).

FIG. 24 shows a graph showing the ffect of Cr⁶⁺ (5 min preincubation) on aequorin light emission in response to the addition of external CaCl₂ (5 mM). Results represent mean±SE.

FIG. 25 shows a graph showing the effect of Cr⁶⁺ (5 min preincubation) on [Ca²⁺]_{c} in response to the addition of external CaCl₂ (5 mM). Results represent mean±SE.

General Cytotoxicity

pure chemicals and chemical mixtures can be tested for their toxicity using aequorin-expressed fungi. Procedure:
i. Add compound(s) of interest to fungus
ii. Monitor [Ca²⁺]_{c} for 5 min
iii. Then stimulate fungus with mechanical perturbation, hypo-osmotic, hyper-osmotic shock
iv. Monitor [Ca²⁺]_{c} for further 5 min

The parameter to be assessed is [Ca²⁺]_{c} final resting level. If [Ca²⁺]_{c} resting level is still elevated more then 50% after the 11 min measurements the compound(s) are toxic. The level of toxicity can be assessed by subsequent monitoring of [Ca²⁺]_{c} for several hours. The longer the [Ca²⁺]_{c} concentration is out of normal the more toxic the compound(s) are. This way there is no need for complicated software and this type of approach is ideally suitable for binary answer, based on 1 parameter.

FIG. 16 shows a graph indicating the effect of 6 environmental samples on [Ca²⁺]_{c}. The graph indicates that sample 006 is toxic as the [Ca²⁺]_{c} final resting level is increased by more than 150% compared with the control.

Another parameter for the analysis of general toxicity is the total amount of [Ca²⁺]_{c} emitted. Based on this parameter it is very easy to build dose response curves (see FIG. 21).

High Information Multiparameters Analysis

In cases when the binary answer is not sufficient aequorin-based biosensor can produce much more detailed data characterising not only the general cytotoxicity but also penetrability (by analysing the time between administration of the compound to the point when [Ca²⁺]_{c} starts to increase) and modes-of-action of the compounds (by comparing the profile of [Ca²⁺]_{c} changes of the compound(s) of interest to the library of profiles). If the mode-of-action of the compound(s) of interest is unique and unknown than the present invention can suggest whether the compound(s) causes the permeabilization of the membrane, opening of ion channels or the alteration in behaviour of Ca²⁺ carriers. This approach is ideally suitable for analysis of combinations of compounds.

This approach can be used for both pollutants monitoring as previously described but also for the analysis of drug toxicity. e.g. ibuprofen and verapamil. FIGS. 17 and 18 show the effect of ibuprofen and verapamil analogues on the [Ca²⁺]_{c} and the table shown in FIG. 19 further summarises the profiles of the ibuprofen and verapamil analogues.

Profiling Compounds of Interest and Creating the Libraries of Fingerprints of Compounds

The present invention is ideally suitable for creating the library of profiles for certain substances. These profiles are unique to a compound with the particular mode-of-action. Also they are unique to the strain of fungus used, which allows creating very details and reproducible fingerprint of a particular compound using the present invention. The profiles can be created with different physico-chemical stimuli (e.g. mechanical perturbation, hypo-osmotic, hyper-osmotic shock, cold shock, heat shock, pH shock). These fingerprints can be programmed into the software and any compounds or mixtures of interest can be screened to match the desired fingerprint.

Procedure to create the fingerprint:
Monitor initial [Ca²⁺]_{c} resting level for 1 min
Add compound(s) of interest to fungus
Monitor [Ca²⁺]_{c} for 5 min
Then stimulate fungus with mechanical perturbation, hypo-osmotic, hyper-osmotic shock
Monitor [Ca²⁺]_{c} for further 5 min
Based on the data obtained the following parameters can be quantified for each [Ca²⁺]_{c} increase occurring during the experiment.
   Lag time
   Rise time
   Amplitude absolute
   Amplitude relative
   Length of transient (LT₂₀, LT₅₀, LT₈₀)
   Initial [Ca²⁺]_{c} level
   Final [Ca²⁺]_{c} resting level
   Recovery time
   Total concentration of [Ca²⁺]_{c}
Above 6 steps can be performed on different strains
Compound can be tested at different concentrations

Considering the nature of the experiment the minimum number of parameters produced by one compound at a particular concentration on one fungal strain is equal 22.

### Analysis of Food and Drink Products for the Presence of Mycotoxins

Fungi transformed with aequorin gene can be also used for the analysis of food and drink products for the presence of mycotoxins since these toxins affect [Ca²⁺]_{c}. Examples of such effects are shown in FIG. 20 where the effects of cyclopiazonic acid (CPA) and KP4 (mycotoxins produced by Ustilago spp) are summarised.

Cosmetics Safety Testing

Since EU regulations forbid the use of animal testing for cosmetics industry the manufacturers are looking at the alternative methods to assess the effect of new products. As the present invention is ideally suited for analysis of not only pure compounds but also their mixtures, it could be used for analysis of the safety of novel cosmetic products. The present invention is also ideal for a long term monitoring of the effects of compounds (up to 96 h), which therefore allows analysis of the longer-term toxicity than bacterial biosensors. The present invention is also suitable for use on different substrates such as solid and liquid supports.

Identification of Different Fungal Strains

It has been found that each particular compound produces a different fingerprint when added to different fungal species. This can be used to diagnose the unknown fungus.

Procedure:
The fungus can be either transformed with the recombinant aequorin gene or can be injected with the active aequorin.
Then this fungus can be subjected to a range of the antifungal drugs, profiles of which have already been created.
Obtained profiles can be compared with the library of the fingerprints and this way the fungal species can be identified.

FIGS. 22a, b and c show that 5 known antifungal drugs (ketoconazole, clotrimazole, amphotericin B, nystatin and filipin) caused a different [Ca²⁺]_{c} response in 3 different species of Aspergillus (A. nidulans, A. niger, A. awamori).

Optimisation of the Current Antifungal Treatments

In order to administer drugs in the best possible way it is important to determine no effect concentration and dose response curves, and frequency for administration of drugs. Also, in view of the developing resistance of fungus and other eukaryotes to currently available drugs, clinicians are looking into using combination of drugs. The present invention is ideally suitable for such studies.

### Identification of Compounds Which Would Prevent Fungal Growth on Plastics, Metals and Other Materials

Since the present invention is suitable for long term measurements it is possible to monitor the development and growth of fungi on different materials and plastics treated with different agents. It is possible to monitor the state of fungal physiology by subjecting the organism to different physico chemical treatments and analysis of the profiles obtained.

Although the invention has been particularly shown and described with reference to particular examples, it will be understood by those skilled in the art that various changes in the form and details may be made therein without departing from the scope of the present invention.

1. A method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
determining whether the amount of light is above or below a defined threshold at the time of exposure.

2. A method as in claim 1 wherein the eukaryote is a fungi.

3. A method as in claim 2 wherein the fungi is a filamentous fungi.

4. A method as in claim 2 wherein the fungi is of the Aspergillus species.

5. A method as in claim 1 wherein the eukaryote is a mammalian cell.

6. A method as in claim 1 wherein the eukaryote is a plant cell.

7. A method as in claim 1 wherein the test sample comprises a toxicant.

8. A method as in claim 1 wherein the light emitting Ca²⁺ regulated photoprotein gene is a recombinant gene.

9. A method as in claim 1 wherein the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

10. A method as in claim 1 wherein the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

11. A method as in claim 1 wherein the light emitting Ca²⁺ regulated photoprotein gene is an aequorin gene.

12. A method as in claim 1 wherein the light emitting Ca²⁺ regulated photoprotein gene is a recombinant aequorin gene.

13. A method as in claim 1 wherein the light that is measured is in the form of luminescence.

14. A method as in claim 1 wherein the test sample is added in advance of the application of a stimulus to the test sample.

15. A method as in claim 14 wherein the stimulus is at least one or more from the group comprising; mechanical perturbation, hypo-osmotic shock, change in external calcium chloride concentration, temperature shock and pH shock.

16. A method as in claim 14 wherein the test sample is added 1 minute to 1 prior to the application of the stimulus.

17. A method as in claim 14 wherein the test sample is added 5 prior to the application of the stimulus.

18. A method as in claim 14 wherein the test sample is added 30 minutes prior to the application of the stimulus.

19. A method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
determining whether the amount of light is above a defined threshold at a specified time after the time of exposure.

20. A method as in claim 19 which comprises the step of determining whether the amount of light is below a defined threshold.

21. A method as in claim 19 wherein the specified time after the time of exposure is 11 minutes.

22. A method as in claim 19 wherein the eukaryote is a fungi.

23. A method as in claim 22 wherein the fungi is a filamentous fungi.

24. A method as in claim 22 wherein the fungi is of the Aspergillus species.

25. A method as in claim 19 wherein the eukaryote is a mammalian cell.

26. A method as in claim 19 wherein the eukaryote is a plant cell.

27. A method as in claim 19 wherein the test sample comprises a toxicant.

28. A method as in claim 19 wherein the light emitting Ca²⁺ regulated photoprotein gene is a recombinant gene.

29. A method as in claim 19 wherein the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

30. A method as in claim 19 wherein the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

31. A method as in claim 19 wherein the light emitting Ca²⁺ regulated photoprotein gene is an aequorin gene.

32. A method as in claim 31 wherein the light emitting Ca²⁺ regulated photoprotein gene is a recombinant aequorin gene.

33. A method as in claim 19 wherein the light that is measured is in the form of luminescence.

34. A method as in claim 19 wherein the test sample is added in advance of the application of a stimulus to the test sample.

35. A method as in claim 34 wherein the stimulus is at least one or more from the group comprising; mechanical perturbation, hypo-osmotic shock, change in external calcium chloride concentration, temperature shock and pH shock.

36. A method as in claim 34 wherein the test sample is added 1 minute to 1 prior to the application of the stimulus.

37. A method as in claim 34 wherein the test sample is added 5 minutes prior to the application of the stimulus.

38. A method as in claim 34 wherein the test sample is added 30 minutes prior to the application of the stimulus.

39. A method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
and comparing the light measurement data with a bank of known toxicity reference data.

40. A method as in claim 39 wherein the method comprises the step of determining whether the amount of light is below a defined threshold.

41. A method as in claim 39 wherein the specified time after the time of exposure is 11 minutes.

42. A method as in claim 39 wherein the eukaryote is a fungi.

43. A method as in claim 42 wherein the fungi is a filamentous fungi.

44. A method as in claim 42 wherein the fungi is of the Aspergillus species.

45. A method as in claim 39 wherein the eukaryote is a mammalian cell.

46. A method as in claim 39 wherein the eukaryote is a plant cell.

47. A method as in claim 39 wherein the test sample comprises a toxicant.

48. A method as in claim 39 wherein the light emitting Ca²⁺ regulated photoprotein gene is a recombinant gene.

49. A method as in claim 39 wherein the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

50. A method as in claim 39 wherein, the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

51. A method as in claim 39 wherein the light emitting Ca²⁺ regulated photoprotein gene is an aequorin gene.

52. A method as in claim 39 wherein the light emitting Ca²⁺ regulated photoprotein gene is a recombinant aequorin gene.

53. A method as in claim 39 wherein the light that is measured is in the form of luminescence.

54. A method as in claim 39 wherein the test sample is added in advance of the application of a stimulus to the test sample.

55. A method as in claim 54 wherein the stimulus is at least one or more from the group comprising; mechanical perturbation, hypo-osmotic shock, change in external calcium chloride concentration, temperature shock and pH shock.

56. A method as in claim 54 wherein the test sample is added 1 minute to 1 prior to the application of the stimulus.

57. A method as in claim 54 wherein the test sample is added 5 minutes prior to the application of the stimulus.

58. A method as in claim 54 wherein the test sample is added 30 minutes prior to the application of the stimulus.

59. A method as in claim 39 wherein the method is used to determine the amount of toxicant in the sample.

60. A method as in claim 39 wherein the method is used to identify the toxicant in the sample.

61. A method of determining the presence of a toxicant in a test sample, comprising the steps of;
exposing a eukaryote that has been transformed with a light emitting Ca²⁺ regulated photoprotein gene to a test sample
measuring the light produced by the transformed cell/organism
converting the light data into a cytosolic free calcium ion concentration trace,
and comparing at least one parameter of the cytosolic free calcium ion concentration trace with a bank of known toxicity reference data.

62. A method as in claim 61 wherein the method comprises the step of determining whether the amount of light is below a defined threshold.

63. A method as in claim 61 wherein the eukaryote is a fungi.

64. A method as in claim 63 wherein the fungi is a filamentous fungi.

65. A method as in claim 63 wherein the fungi is of the Aspergillus species.

66. A method as in claim 61 wherein the eukaryote is a mammalian cell.

67. A method as in claim 61 wherein the eukaryote is a plant cell.

68. A method as in claim 61 wherein the test sample comprises a toxicant.

69. A method as in claim 61 wherein the light emitting Ca²⁺ regulated photoprotein gene is a recombinant gene.

70. A method as in claim 61 wherein the light emitting Ca²⁺ regulated photoprotein gene is selected from the group comprising;
aequorin gene
halistaurin (mitrocomin) gene
phialidin (clytin) gene
obelin gene
mnemiopsin gene
berovin gene

71. A method as in claim 61 wherein the light emitting Ca²⁺ regulated photoprotein gene may be a functional homologue of a gene selected from the group comprising;
aequorin gene
halistaurin (mitrocomin) gene
phialidin (clytin) gene
obelin gene
mnemiopsin gene
berovin gene

72. A method as in claim 61 wherein the light emitting Ca²⁺ regulated photoprotein gene is an aequorin gene.

73. A method as in claim 61 wherein the light emitting Ca²⁺ regulated photoprotein gene is a recombinant aequorin gene.

74. A method as in claim 61 wherein the light that is measured is in the form of luminescence.

75. A method as in claim 61 wherein the test sample is added in advance of the application of a stimulus to the test sample.

76. A method as in claim 75 wherein the stimulus is at least one or more from the group comprising; mechanical perturbation, hypo-osmotic shock, change in external calcium chloride concentration, temperature shock and pH shock.

77. A method as in claim 75 wherein the test sample is added 1 minute to 1 prior to the application of the stimulus.

78. A method as in claim 75 wherein the test sample is added 5 minutes prior to the application of the stimulus.

79. A method as in claim 75 wherein the test sample is added 30 minutes prior to the application of the stimulus.

80. A method as in claim 61 wherein light is measured for between 1 minute and 5 hours following the application of the stimulus.

81. A method as in claim 61 wherein light is measured for between 1 minute and 96 hours following the application of the stimulus.

82. A method as in claim 61 wherein light is measured for 5 minutes following the application of the stimulus.

83. A method as in claim 61 wherein the cytosolic free calcium ion trace is a plot of the cytosolic free calcium ion concentration against time.

84. A method as in claim 61 wherein the parameter is at least one or more selected from the group comprising;
lag time
rise time
absolute amplitude
relative amplitude
Length of transient at 20%, 50% and 80% of maximum amplitude
number of cytosolic free calcium ion concentration increases
percentage increase in final cytosolic free calcium ion concentration resting level
percentage increase in recovery time
percentage increase in pre-stimulating cytosolic free calcium ion concentration resting level
total concentration of calcium

85. A method as in claim 61 wherein the method is used to determine the amount of toxicant in the sample.

86. A method as in claim 61 wherein the method is used to identify the toxicant in the sample.
